(19) United States
(12) Patent Application Publication
(10) Pub. No.: US 2023/0218235 A1
(43) Pub. Date: Jul. 13, 2023

(54) STABILIZATION ASSEMBLY FOR HEAD-MOUNTED DEVICE

(71) Applicant: X Development LLC, Mountain View, CA (US)

(72) Inventor: Phillip Yee, San Francisco, CA (US)

(21) Appl. No.: 17/570,739

(22) Filed: Jan. 7, 2022

(51) Int. Cl. A61B 5/00; A61B 5/055

(52) U.S. Cl. CPC A61B 5/702; A61B 5/055

(57) ABSTRACT

Disclosed are an apparatus and a stabilization assembly for supporting a device on a human head, including: a first frame having an annular shape with a first diameter; and a second frame having an annular shape with a second diameter, the second diameter being less than the first diameter. The second frame is concentric with the first frame and is mechanically coupled to the first frame by a pivot support that permits rotation of the second frame relative to the first frame. A tripod mount is mechanically coupled to the first frame, the tripod mount including legs configured to rest on the human head during use of the stabilization assembly. The stabilization assembly positions the device against the human head during use. The tripod mount includes three legs, each leg having a U-shape. For each leg, ends of the U-shaped leg connect to the first frame.

### FIELD

This specification relates to mechanical devices for use in medical imaging machines.

### BACKGROUND

Direct stimulation to the human brain has been shown to have many positive effects, anecdotally and subjectively. This has been a subject of study in many areas of neuroscience, psychology, medication therapy, direct stimulation therapy, and other applications. To provide direct stimulation to the brain, a device is placed against the human head.

### SUMMARY

Disclosed is a stabilization assembly for a head-mounted device. The stabilization assembly is a hardware apparatus that can hold devices accurately on the head. In this way, the assembly can be placed to affect particular parts of the head, e.g., external, skin, skull, or ear canal. The assembly can also be placed to affect particular lobes of the brain, e.g., frontal, occipital, or temporal. The assembly is configured to hold a device to the head, such that the device maintains contact with the head. The assembly is also configured to maintain accurate positioning by aiming the device to a precise location, and keeping the device stable on the head.

An example device that can be held by the assembly is an ultrasonic transducer. An ultrasonic transducer emits ultrasonic waves along to a desired depth into the head. The assembly can also be configured to mount any small device to a human head. Example devices can include diagnostic equipment, sensors and instruments, therapeutic devices, comfort items, actuators, massagers, and other types of devices.

The assembly allows for a wide range for device placement on the human head. For example, the assembly can hold the device to the rear, sides, top, or frontal areas of the head. Flexibility of placement of the assembly is enabled by a tripod support. The tripod support can be placed securely on any part of a head, and is configured to fit different head shapes, head sizes, and irregular contours.

The assembly includes a headset, the position of which can be adjusted coarsely and finely. Coarse adjustment can include adjustment of a headband that is secured to the head, e.g., around the forehead of the user. Coarse adjustment can also include adjustment of multiple straps that are configured to wrap around the chin of the user.

The assembly includes two concentric circular frames arranged in a gimbal-like configuration. An inner frame is configured to hold the device, while an outer frame connects to the tripod support. Fine adjustment can include adjusting the pitch or roll of the inner frame relative to the outer frame. The assembly can include adjustment knobs to permit rotational adjustment of the inner frame. Fine adjustment can be used to accurately and precisely adjust the centerline axis of the device to point towards the desired target of the head. Find adjustment can be performed in conjunction with the user of visualization and aiming technologies to confirm proper trajectory. For example, infrared cameras can locate the positions of the head and of the device, providing a real-time trajectory of transmission of the device.

The assembly is compact and comfortable, enabling the user to wear the device for extended periods of time inside a magnetic resonance imaging (MM) machine. The assembly is MRI-compatible. The assembly is composed of non-metal materials and non-ferromagnetic materials. Thus, the assembly does not cause sign interference in an MRI environment. This enables the direct correlation of input or stimulation to observed effects, such as brain activity observed as a result of ultrasonic input.

In one general aspect, a stabilization assembly for supporting a device on a human head, including: a first frame having an annular shape with a first diameter; and a second frame having an annular shape with a second diameter, the second diameter being less than the first diameter. The second frame is concentric with the first frame and is mechanically coupled to the first frame by a pivot support that permits rotation of the second frame relative to the first frame. The stabilization assembly includes a tripod mount mechanically coupled to the first frame, the tripod mount including legs configured to rest on the human head during use of the stabilization assembly. The stabilization assembly positions the device against the human head during use.

These and other embodiments may each optionally include one or more of the following features, alone or in combination. In some implementations, the tripod mount includes three legs, each leg having a U-shape. For each leg, ends of the U-shaped leg connect to the first frame.

In some implementations, each leg includes a first segment encapsulated by a cushioned tube, the first segment being configured to contact the human head when the stabilization assembly is worn by the human.

In some implementations, each leg includes a second segment extending approximately parallel to the first segment, the second segment being configured to anchor a strap for connecting the tripod mount to a headband.

In some implementations, the stabilization assembly includes an adjustable headband configured to wrap around the human head; and a plurality of adjustable straps connecting the tripod mount to the adjustable headband.

In some implementations, the stabilization assembly includes one or more chinstraps coupled to the headband. The one or more chinstraps are configured to secure the headband to the chin of the human head.

In some implementations, the plurality of adjustable straps are configured to hold the device in place at any of the occipital region, the frontal region, the temporal region, or the parietal region of the human head.

In some implementations, the first frame, the second frame, and the tripod mount are formed from non-metal materials.

In some implementations, the stabilization assembly includes a first adjustment knob for adjusting a rotation of the second frame relative to the first frame along a first axis; and a second adjustment knob for adjusting the rotation of the second frame relative to the first frame along a second axis.

In some implementations, the device has a cylindrical shape, an outer perimeter of the device fitting within an inner perimeter of the second frame.

In some implementations, the second frame includes an aperture extending between the inner perimeter of the second frame and an outer perimeter of the second frame, the aperture configured to permit passage of a power cable for the device.

In some implementations, the second frame includes a plurality of capsules spaced equidistantly around a perimeter of the second frame.

In some implementations, the plurality of capsules are each configured to hold a softgel capsule that is visible to a magnetic resonance imaging system.

In some implementations, the stabilization assembly is configured to support the device on the human head while the human is prone.

In some implementations, the stabilization assembly is configured to support the device on the human head while the human head is inside an MRI envelope.

In some implementations, the device is coupled to a water bladder, and the stabilization assembly is configured to support the device on the human head in a position such that when the water bladder is inflated, the device contacts the human head.

In some implementations, the device includes one or more of a transducer, a diagnostic medical device, a sensor, an instrument, a therapeutic device, an actuator, or a massager.

In another general aspect, an apparatus for positioning a device against a human head includes: a first frame having an annular shape with a first diameter; and a second frame having an annular shape with a second diameter, the second diameter being less than the first diameter. The second frame is concentric with the first frame and is mechanically coupled to the first frame by a support that permits rotation of the second frame relative to the first frame. The apparatus includes: a tripod mount mechanically coupled to the first frame, the tripod mount including legs configured to rest on the human head during use of the apparatus; an adjustable headband configured to wrap around the human head during use of the apparatus; a plurality of adjustable straps connecting the tripod mount to the adjustable headband; and one or more chinstraps coupled to the headband and configured to secure the headband to the chin of the human head during use of the apparatus.

These and other embodiments may each optionally include one or more of the following features, alone or in combination. In some implementations, the tripod mount includes three legs, each leg having a U-shape. For each leg, ends of the U-shaped leg connect to the first frame.

In some implementations, each leg includes: a first segment encapsulated by a cushioned tube, the first segment being configured to contact the human head when the apparatus is worn by the human; and a second segment extending approximately parallel to the first segment, the second segment being configured to anchor at least one of the plurality of adjustable straps.

Among other advantages, embodiments feature improved accuracy and preciseness of mounting devices to a human head. The disclosed stabilization assembly is small and comfortable for wear on a human head. When worn by a user inside an Mill machine, the stabilization assembly permits the user to lie comfortably with his or her head in a natural position. The disclosed stabilization assembly is easy to install on a human head and is easy to adjust, both coarsely and finely. The assembly can be fine-tuned to accurately pinpoint a target, and the stable position will not drift, even during movement of the head. The stabilization assembly includes capsule holders that are configured to hold softgel capsules, such as capsules containing Vitamin A or Vitamin E, that can be viewed in MM images to enable a user to view the orientation of the assembly inside the MRI machine. The non-metal assembly can be worn by a user inside the MM machine without interfering with the MRI images.

The details of one or more implementations are set forth in the accompanying drawings and the description, below. Other potential features and advantages of the disclosure will be apparent from the description and drawings, and from the claims.

### BRIEF DESCRIPTION OF THE DRAWINGS

FIG. 1 illustrates an example stabilization assembly worn by a patient in an imaging envelope.

FIGS. 2A and 2B are cross-sectional views of the example stabilization assembly of FIG. 1.

FIGS. 3A and 3B are perspective views of the example stabilization assembly of FIG. 1.

FIG. 4 illustrates an example apparatus for securing the stabilization assembly to a human head.

FIGS. 5A and 5B are perspective views of an example device that can be positioned against the human head using the stabilization assembly.

Like reference numbers and designations in the various drawings indicate like elements. The components shown here, their connections and relationships, and their functions, are meant to be examples only, and are not meant to limit the implementations described and/or claimed in this document.

### DETAILED DESCRIPTION

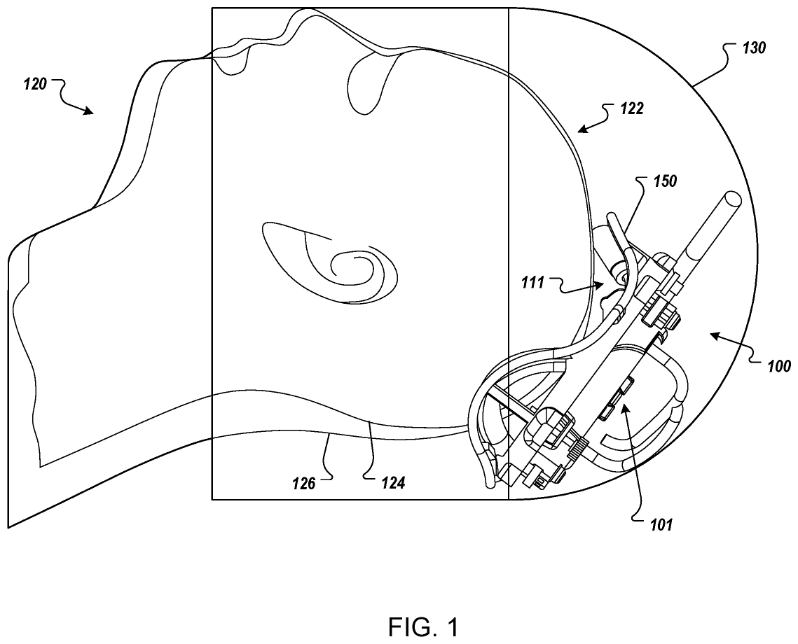

FIG. 1 illustrates an example stabilization assembly 100 (“assembly 100”) that can be worn by a patient, such as patient 120. The patient 120 is a human being examined within an imaging envelope 130. The assembly 100 can be used to support a device 101 on a human head 122 of the patient 120. The assembly 100 positions the device 101 against the human head 122 during use.

In some examples, the assembly is configured to support the device 101 on the human head 122 while the human head 122 is inside the image envelope 130. The imaging envelope 130 can be, for example, an MRI envelope. The device 101 can be, for example, a transducer such as an ultrasonic transducer. Within the imaging envelope 130, imaging can be performed using ultrasound data or other imaging data, such as EEGs and MRIs.

The assembly can be worn by patients with various head sizes. For example, the assembly 100 can be worn by children with smaller head sizes and by adults with larger head sizes. To permit the assembly 100 to fit various head sizes, the assembly 100 includes a tripod mount 150 that can fit on spherical or near spherical shapes of various sizes. The tripod mount 150 rests on the head 122 such that a part of the head 122 extends into a cavity 111 formed by the tripod. FIG. 1 shows two different head sizes, e.g., head size 124 and head size 126. The assembly 100 fits on both head sizes 124 and 126. Accordingly, the assembly 100 can be considered to be “one size fits all” or “one size fits most.”

The assembly 100 can be easily placed on the head 122 of the patient 120, and can be maintained in a steady position when worn by a mobile patient. The patient 120 may be able to sit up, lie down, and walk around while wearing the assembly 100. In some examples, the assembly 100 is configured to support the device 101 on the human head 122 while the patient 120 is prone. The assembly is configured such that the patient 120 can maintain the head 122 in a natural, comfortable position while wearing the assembly 100 and lying prone.

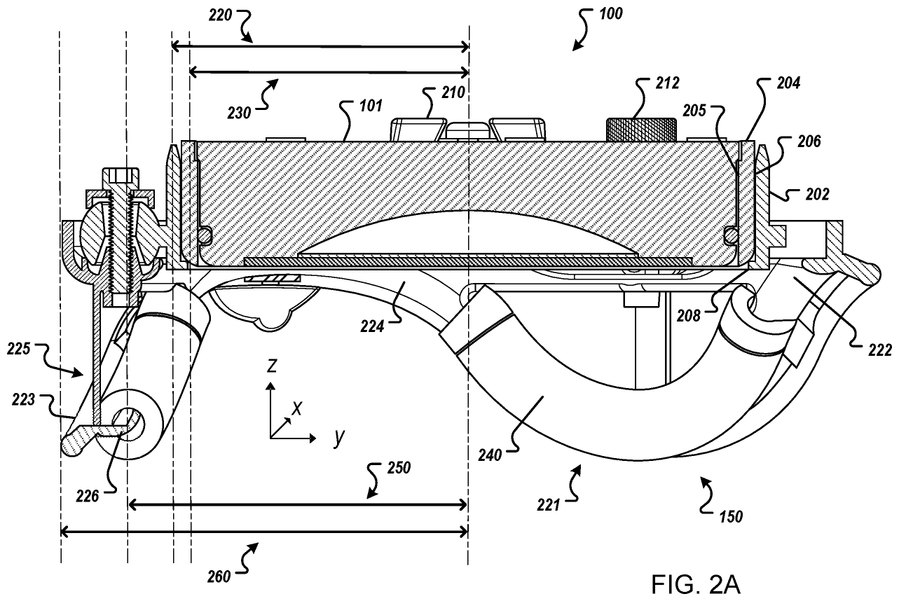

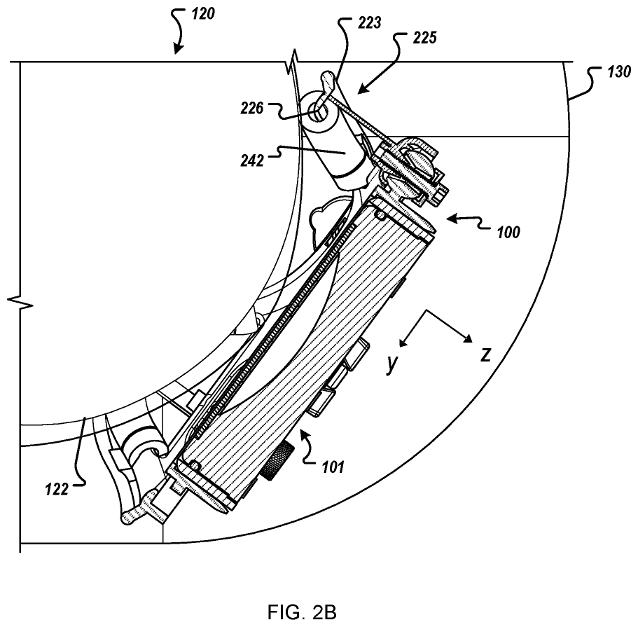

FIGS. 2A and 2B are cross-sectional views of the assembly 100. FIG. 2A shows a cross-sectional view of the assembly 100 and the device 101. FIG. 2B shows a cross-sectional view of the assembly 100 and the device 101 worn by the patient 120 in the imaging envelope 130. FIGS. 2A. Cartesian coordinate directions are shown in FIGS. 2A and 2B for reference.

Referring to FIG. 2A, the assembly 100 includes a first frame 202 having an annular shape with a first radius 220 in the y-direction. The first radius 220 can be, for example, 55 mm or less, 53 mm or less, or 51 mm or less.

The assembly 100 includes a second frame 204 having an annular shape with a second radius 230 in the y-direction. The second radius 230 is less than the first radius 220. The second frame 204 is concentric with the first frame 202. The second frame 204 has an inner perimeter 205.

In some examples, the first frame 202 and the second frame 204 each have a circular shape in the x-y plane. In some examples, the first frame 202 and the second frame 205 each have an oval or elliptical shape in the x-y plane.

In some examples, the device 101 has a cylindrical shape in the x-y plane. An outer perimeter of the device 101 fits within the inner perimeter 205 of the second frame 204. The second frame 204 can include a rim 206 along an inner surface of the second frame 204. The rim 206 can hold the device 101 in place within the inner perimeter 205 of the second frame 204. For example, the device 101 can include a groove that permits the device 101 to snap into place within the second frame 204. The device 101 can have radius in the x-y plane of, for example, 53 mm or less, 52 mm or less, or 50 mm or less. The device 101 can have a thickness in the z-direction of, for example, 25 mm or less, 23 mm or less, or 21 mm or less.

The assembly 100 can include retainer hooks 210. The retainer hooks 210 can be used to secure the device 101 within the second frame 204 and prevent the device 101 from falling out of the second frame 204 along the z-direction away from the tripod mount 150. The assembly 100 can also include a retainer screw 212 for controlling the device retainer hooks 210, e.g., by locking and unlocking the retainer hooks 210. The retainer hooks can secure the device 110, e.g., by cinching the device 101 to the assembly 100. The second frame 204 includes a hard stop 208. The hard stop 208 can be a ridge along the inner perimeter of the second frame 204. The hard stop 208 can support the device 101 and prevents the device 101 from falling out of the second frame 204 along the z-direction towards the tripod mount 150.

In some examples, the second frame 204 can be removable and/or replaceable from the assembly 100. For example, the second frame 204 can be configured to support a first device. A different second frame can be configured to support a different, second device. The second frame 204 can be removed from the assembly 100 and replaced with the different second frame, such that the assembly 100 including the different second frame can support the second device. The second frame 204 and the different second frame can have different shapes, sizes, and securing mechanisms for supporting different types of devices.

The assembly 100 has a thickness in the z-direction. The thickness can be measured from a top of the second frame 204 in the z-direction to a bottom of a leg of the tripod mount in the z-direction. The thickness of the assembly 100 can be, for example, 70 mm or less, 65 mm or less, or 60 mm or less.

The tripod mount 150 is mechanically coupled to the first frame 202. The tripod mount 150 includes three legs configured to rest on the human head during use of the assembly 100. Each leg has a U-shape, and ends of each U-shaped leg connect to the first frame 202. For example, a first end 222 of a first leg 221 connects to the first frame 202. A second end 224 of the first leg 221 also connects to the first frame 202.

The tripod mount 150 can also be described as being a “wavy” tripod, such that each of the three legs of the tripod connects to each other leg of the tripod in a wave shape. The tripod mount therefore includes three waves, where a crest of each wave is coupled to the first frame, and a trough of each wave rests on the human head when the assembly 100 is in use. The wavy tripod shape permits the assembly to cradle various shapes and sizes of human heads.

Each leg of the tripod mount 150 includes at least two segments. The two segments can each have a U-shape and can be parallel to each other. In some examples, a first segment of the two segments is encapsulated by a cushioned tube, e.g., cushioned tube 240. Referring to FIG. 2B, a first segment 226 of leg 225, encapsulated by cushioned tube 242, is configured to contact the human head 122 when the assembly 100 is worn by the patient 120. The second segment of each leg is configured to anchor a strap for connecting the tripod mount 150 to a headband. For example, a second segment 223 of the leg 225 is configured to anchor a strap for holding the assembly 100 in place on the head 122.

Referring to FIG. 2A, the first segment 226 of the leg 225 has a radius 250, as measured from the center of the assembly in the x-y plane. The radius 250 can be, for example, 62 mm or less, 61 mm or less, or 60 mm or less. The second segment 223 of the leg 225 has a radius 260. The radius 260 can be, for example, 77 mm or less, 76 mm or less, or 75 mm or less.

An outer diameter of the cushioned tube 240 can be, for example, 15 mm or less, 13 mm or less, or 11 mm or less. In some examples, the cushioned tube 240 is formed from a silicone material. For example, the cushioned tube 240 can be a silicone vacuum tube.

In some examples, the first frame, the second frame, and the tripod mount are formed from non-metal materials. For example, the first frame, the second frame, and the tripod mount can be formed from materials including plastic and/or rubber.

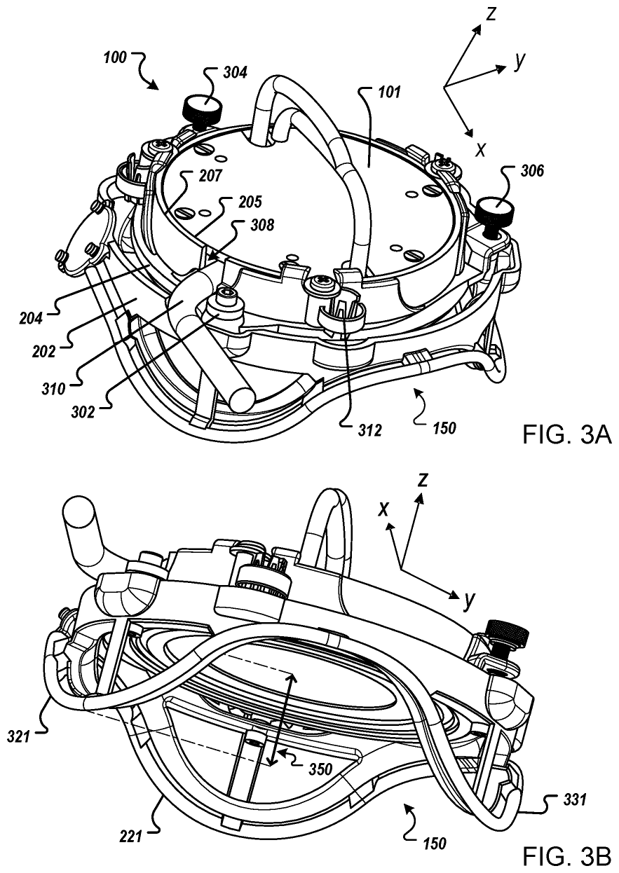

FIGS. 3A and 3B are perspective views of the assembly 100. Cartesian coordinate directions are shown in FIGS. 3A and 3B for reference.

Referring to FIG. 3A, the second frame 204 is concentric with the first frame 202. The second frame 204 is mechanically coupled to the first frame 202 by a pivot support 302. The pivot support 302 can include, for example, a ball pivot joint. The pivot support 302 permits rotation of the second frame 204 relative to the first frame 202. Thus, the second frame 204 and the first frame 202 form a gimbal-like assembly.

The assembly 100 includes a first adjustment knob 304 and a second adjustment knob 306 for making fine adjustments to the position of the second frame 204 relative to the first frame 202. The second frame 204 can be adjusted or rotated, e.g., five degrees or less in any direction. The second frame 204 can be raised, lowered, rotated, or pivoted relative to the first frame 202.

The assembly 100 includes a first adjustment knob 304 for adjusting a rotation of the second frame 204 relative to the first frame 202 along a first axis, e.g., the x-axis. The first adjustment knob 304 can thus be used to adjust a degree of “roll” of the second frame 204 relative to the first frame 202.

The assembly 100 includes a second adjustment knob 306 for adjusting a rotation of the second frame 204 relative to the first frame 202 along a second axis. In some examples, the second axis is perpendicular to the first axis, e.g., the y-axis. The second adjustment knob 306 can thus be used to adjust a degree of “pitch” of the second frame 204 relative to the first frame 202.

The second frame 204 includes an aperture 308 extending between the inner perimeter 205 of the second frame 204 and an outer perimeter 207 of the second frame. The aperture 308 is configured to permit passage of a power cable 310 for the device 101.

The second frame 204 includes capsule holders, e.g., capsule holder 312, spaced equidistantly around a perimeter of the second frame 204. The capsule holders are each configured to hold a softgel capsule that is visible to a magnetic resonance imaging (MRI) system. In some examples, the second frame 204 includes three capsule holders, with each capsule holder being coupled to the outer perimeter 207 of the second frame. The three capsule holders can be spaced equidistantly, e.g., at intervals of one hundred twenty degrees, around the perimeter of the second frame 204. In some examples, the softgel capsule is a Vitamin E or Vitamin A tablet. The softgel capsule includes fatty materials that are visible in an MRI image. A user viewing the MM image can arrange the assembly 100 at an appropriate position on the head 122 using the visible softgel capsules as guides.

Referring to FIG. 3B, the tripod mount 150 includes three legs 221, 321, 331. The tripod mount 150 has a depth 350 from the device 101 to the troughs of the tripod legs in the z-direction. The depth 350 can be, for example, 50 mm or less, 40 mm or less, or 35 mm or less.

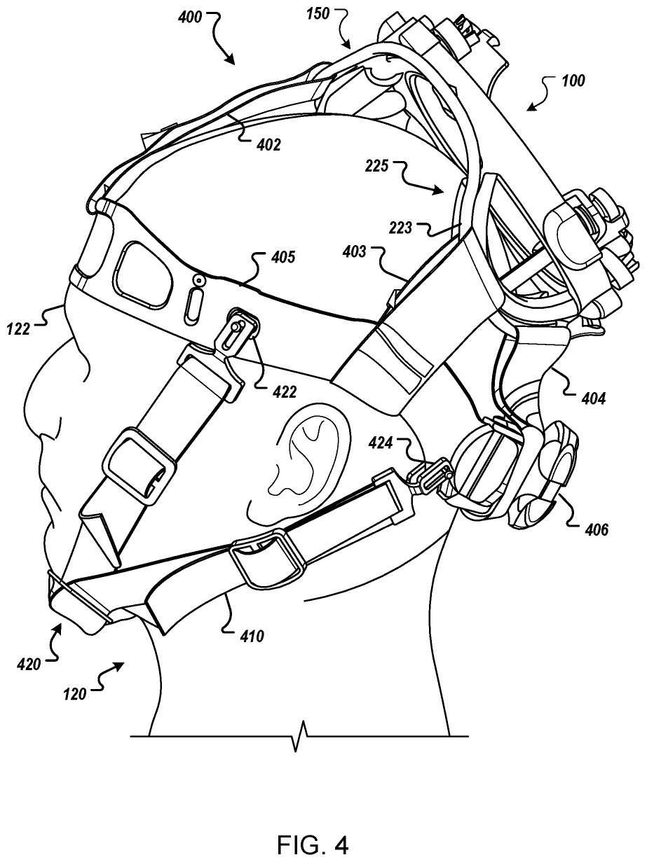

FIG. 4 is an illustration of an example apparatus 400 for securing the assembly 100 to a human head 122. The apparatus includes a headband 405. The headband 405 is similar to a headband of a hardhat. The headband 405 wraps around the forehead of the patient 120. The headband 405 is adjustable by an adjustment knob 406 that can loosen and tighten the headband 405.

The apparatus 400 includes straps 402, 403, and 404. The straps 402, 403, 404 each attach at a first end to the tripod mount 150, and at a second end to the headband 405. Each strap can attach to a second segment of a leg of the tripod mount. For example, the strap 403 and the strap 404 each attach to the second segment 223 of the leg 225. In some examples, two straps attach to each leg of the tripod mount 150. Thus, the apparatus 400 can include six total straps connecting the tripod mount 150 to the headband. In some examples, the apparatus 400 can include more or fewer straps. For example, the apparatus 400 can include one strap attached to each leg of the tripod mount 150, or three straps attached to each leg of the tripod mount 150.

In some examples, the straps 402, 403, 404 are Velcro straps. The straps 402, 403, 404 are adjustable to enable relocation of the assembly 100 relative to the headband 405. For example, the strap 402 can be tightened, and the strap 404 can be loosened, to permit relocation of the assembly from the back of the head 122 towards the forehead of the patient 120, or from the right side of the head 122 towards the left side of the head 122. In some examples, the straps 402, 403, 404, are adjustable to permit placement of the device 101 at any location on the head 122 that is at or above the position of the headband 405. When the assembly 100 is in position, the straps 402, 403, 404 can be tightened to securely fit the assembly 100 to the head 122.

The apparatus 400 includes a chinstrap 410. The chinstrap 410 attaches to the headband on either side of the head 122. The chinstrap 410 is configured to wrap around the chin 420 of the patient. The chinstrap 410 attaches to the headband 405 at attachment points 422, 424 on the left side of the head 122, and at two attachment points on the right side of the head 122 (not shown). In some examples, the chinstrap 410 can attach to the headband 405 at more or fewer attachment points, e.g., at one attachment point on each side of the head or at three attachment points on each side of the head.

In some examples, the apparatus 400 can include two or more chinstraps. The chinstraps are configured to secure the headband 405 to the chin 420 of the head 122. The chinstraps are adjustable, and can be loosened or tightened to securely hold the headband 405 in place.

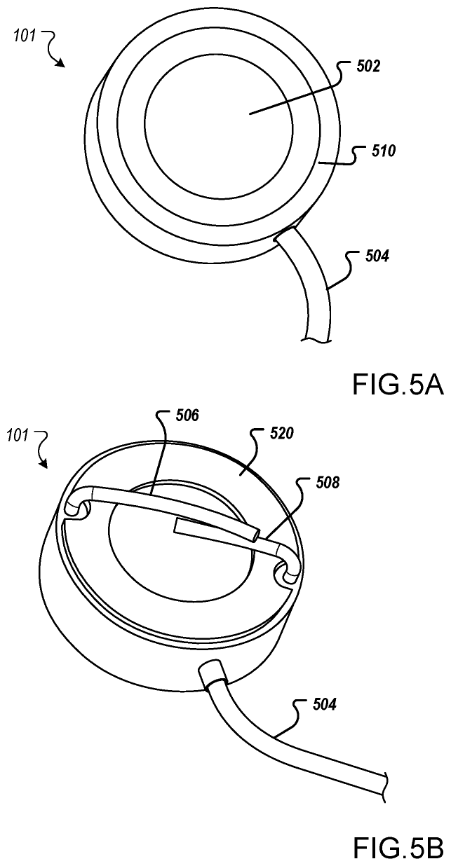

FIGS. 5A and 5B are perspective views of an example device 101 that can be positioned against the human head using the stabilization assembly. FIG. 5A shows a first side 510 of the device 101, and FIG. 5A shows a second side 520 of the device 101 that is opposite the first side 510. The device 101 has a cylindrical, puck-like or disc-like shape. The device 101 can be, for example, a transducer, a diagnostic medical device, a sensor, an instrument, a therapeutic device, an actuator, or a massager.

The device 101 can be coupled to a bladder 502, e.g., a water bladder. The device 101 can be coupled to a bladder inlet 506 and a bladder outlet 508. The bladder inlet 506 is configured to conduct a fluid, e.g., water, to the bladder 502 to inflate the bladder 502. The bladder outlet 508 is configured to conduct the fluid out of the bladder 502.

The assembly 100 is configured to support the device 101 on the human head 122 in a position such that when the bladder 502 is inflated, the device 101 contacts the human head 122. When the assembly 100 holds the device 101 against the head 122, the bladder can be inflated to push the surface of the device 101 against the head 122 so that the device 101 is in contact with the head 122. In some examples, the bladder 502 can be coated with a gel such as an ultrasound gel. In some examples, ultrasound gel can be applied to the device in addition to, or in lieu of, the bladder 502.

In some examples, the device 101 is an electrically powered device. The device 101 can be powered by a power system that is integrated with the device 101 or with the assembly 100. For example, the device 101 or the assembly 100 can include a battery. In some implementations, the device 101 can be powered by a separate power source. In some implementations, the device 101 is connected to a power source by one or more power cables, e.g., power cable 504.

A number of implementations have been described. Nevertheless, it will be understood that various modifications may be made without departing from the spirit and scope of the disclosure.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

What is claimed is:

1. A stabilization assembly for supporting a device on a human head, comprising:
a first frame having an annular shape with a first diameter;
a second frame having an annular shape with a second diameter, the second diameter being less than the first diameter, wherein the second frame is concentric with the first frame and is mechanically coupled to the first frame by a pivot support that permits rotation of the second frame relative to the first frame; and
a tripod mount mechanically coupled to the first frame, the tripod mount comprising legs configured to rest on the human head during use of the stabilization assembly,
wherein the stabilization assembly positions the device against the human head during use.

2. The stabilization assembly of claim 1, wherein the tripod mount comprises three legs, each leg having a U-shape, wherein for each leg, ends of the U-shaped leg connect to the first frame.

3. The stabilization assembly of claim 2, wherein each leg comprises a first segment encapsulated by a cushioned tube, the first segment being configured to contact the human head when the stabilization assembly is worn by the human.

4. The stabilization assembly of claim 3, wherein each leg comprises a second segment extending approximately parallel to the first segment, the second segment being configured to anchor a strap for connecting the tripod mount to a headband.

5. The stabilization assembly of claim 1, comprising:
an adjustable headband configured to wrap around the human head; and
a plurality of adjustable straps connecting the tripod mount to the adjustable headband.

6. The stabilization assembly of claim 5, comprising:
one or more chinstraps coupled to the headband, wherein the one or more chinstraps are configured to secure the headband to the chin of the human head.

7. The stabilization assembly of claim 5, wherein the plurality of adjustable straps are configured to hold the device in place at any of the occipital region, the frontal region, the temporal region, or the parietal region of the human head.

8. The stabilization assembly of claim 1, wherein the first frame, the second frame, and the tripod mount are formed from non-metal materials.

9. The stabilization assembly of claim 1, comprising:
a first adjustment knob for adjusting a rotation of the second frame relative to the first frame along a first axis; and
a second adjustment knob for adjusting the rotation of the second frame relative to the first frame along a second axis.

10. The stabilization assembly of claim 1, wherein the device has a cylindrical shape, an outer perimeter of the device fitting within an inner perimeter of the second frame.

11. The stabilization assembly of claim 10, wherein the second frame comprises an aperture extending between the inner perimeter of the second frame and an outer perimeter of the second frame, the aperture configured to permit passage of a power cable for the device.

12. The stabilization assembly of claim 1, wherein the second frame comprises a plurality of capsules spaced equidistantly around a perimeter of the second frame.

13. The stabilization assembly of claim 12, wherein the plurality of capsules are each configured to hold a softgel capsule that is visible to a magnetic resonance imaging system.

14. The stabilization assembly of claim 1, wherein the stabilization assembly is configured to support the device on the human head while the human is prone.

15. The stabilization assembly of claim 14, wherein the stabilization assembly is configured to support the device on the human head while the human head is inside an MRI envelope.

16. The stabilization assembly of claim 1, wherein:
the device is coupled to a water bladder, and
the stabilization assembly is configured to support the device on the human head in a position such that when the water bladder is inflated, the device contacts the human head.

17. The stabilization assembly of claim 1, wherein the device comprises one or more of a transducer, a diagnostic medical device, a sensor, an instrument, a therapeutic device, an actuator, or a massager.

18. An apparatus for positioning a device against a human head, comprising:
a first frame having an annular shape with a first diameter;
a second frame having an annular shape with a second diameter, the second diameter being less than the first diameter, wherein the second frame is concentric with the first frame and is mechanically coupled to the first frame by a support that permits rotation of the second frame relative to the first frame;
a tripod mount mechanically coupled to the first frame, the tripod mount comprising legs configured to rest on the human head during use of the apparatus;
an adjustable headband configured to wrap around the human head during use of the apparatus;
a plurality of adjustable straps connecting the tripod mount to the adjustable headband; and
one or more chinstraps coupled to the headband and configured to secure the headband to the chin of the human head during use of the apparatus.

19. The apparatus of claim 18, wherein the tripod mount comprises three legs, each leg having a U-shape, wherein for each leg, ends of the U-shaped leg connect to the first frame.

20. The apparatus of claim 19, wherein each leg comprises:
a first segment encapsulated by a cushioned tube, the first segment being configured to contact the human head when the apparatus is worn by the human; and
a second segment extending approximately parallel to the first segment, the second segment being configured to anchor at least one of the plurality of adjustable straps.
